(19) United States
(12) Patent Application Publication
(10) Pub. No.: US 2026/0191699 A1
(43) Pub. Date: Jul. 9, 2026

(54) ABSORBENT ARTICLE WITH IMPROVED FIT AND CONFORMITY

(71) Applicant: Kimberly-Clark Worldwide, Inc., Neenah, WI (US)

(72) Inventors: Juliana Rodrigues Calil, São Paulo (BR); Stephanie Anne-Genevieve Riemer, Hortonville, WI (US); Linsey Ann Griffin, Saint Paul, MN (US)

(21) Appl. No.: 19/132,574

(22) PCT Filed: Dec. 2, 2022
(86) PCT No.: PCT/US2022/080807

§ 371 Date: May 23, 2025

(51) Int. Cl. A61F 13/49

(52) U.S. Cl. CPC A61F 13/49006; A61F 13/49015

(57) ABSTRACT

An absorbent article includes a chassis extending in a longitudinal direction and at least one ear attached to, and extending outwardly from, the chassis. The at least one ear is formed of a fabric adapted to stretch such that the at least one ear has a stretch direction. The at least one ear is attached to the chassis such that the stretch direction of the at least one ear is oriented relative to the longitudinal direction at an oblique stretch angle when the absorbent article is oriented in a flat, laid-ont position and in an uncontracted state.

### FIELD

The present technology relates to absorbent articles intended to be worn, and more particularly, and not by way of limitation, to diapers having an improved geometry for better fit and conformity to the wearer.

### BACKGROUND

Many absorbent articles intended for personal wear, such as diapers, training pants, feminine hygiene products, adult incontinence products, bandages, medical garments and the like are designed to be sufficiently absorbent to absorb moisture from liquid body exudates (including urine, menses, blood, etc.) and wick them away from the wearer to reduce skin irritation caused by prolonged wetness exposure. Diapers, as an example, are typically placed and secured on a wearer using a set of primary fastening tabs, such as adhesive tabs or mechanical (e.g., hook or loop) fastening system tabs, and left in place to absorb exudates as well as to contain fecal waste.

Diapers are usually characterized as disposable or reusable. Disposable diapers have dominated the market for decades and are commonly provided in customized sizes to fit children in various stages of growth based on weight and height. Once a child outgrows a smaller diaper, the next size range of diapers must be bought.

In contrast to the single-use nature of disposable diapers, reusable diapers may be wholly or partially reusable such that the general construction can be used again and again and often are intended to fit children in different stages of growth. Reusable cloth diapers existed long before disposable diapers, but they were generally ill fitting, bulky, and often failed to effectively contain liquid and other waste. Improvements have been made to reusable diapers to address the drawbacks of traditional cloth diapers. For example, many reusable diapers include a durable outer shell in which is positioned a disposable or reusable absorbent component that may be removed and replaced and that is better able to absorb and contain bodily fluids. Moreover, reusable diapers have been shaped to better conform to the wearer's anatomy and often include elastic in the waistband and leg openings to prevent leakage more effectively. Even with such improvements, existing reusable diapers still have issues with fit, comfort, and performance when compared to disposable diapers. Such issues arise, in part, from a desire to provide reusable diapers in a size that is intended, or that can be adapted, to universally fit wearers in different stages of growth. Thus, further improvements are needed to allow reusable diapers to accommodate a child, particularly as the child grows.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, an absorbent article includes a chassis extending in a longitudinal direction and at least one ear attached to, and extending outwardly from, the chassis. The at least one ear is formed of a fabric adapted to stretch such that the at least one ear has a stretch direction. The at least one ear is attached to the chassis such that the stretch direction of the at least one ear is oriented relative to the longitudinal direction at an oblique stretch angle when the absorbent article is oriented in a flat, laid-out position and in an uncontracted state.

According to certain embodiments, an absorbent article includes a chassis extending in a longitudinal direction and having a first lateral side and an opposing second lateral side. A first ear is attached to, and extends outwardly from, the first lateral side, and a second ear is attached to, and extends outwardly from, the second lateral side. The first car and the second ear are each formed of a fabric adapted to stretch in at least one of a machine direction or a cross-machine direction such that the first ear has a first ear stretch direction and the second ear has a second ear stretch direction, where each of the first ear stretch direction and the second ear stretch direction is parallel to one of the machine direction or the cross-machine direction of the fabric. The first ear is attached to the first lateral side such that the first ear stretch direction is oriented relative to the longitudinal direction at a first ear stretch angle when the absorbent article is oriented in a flat, laid-out position and in an uncontracted state, and the second ear is attached to the second lateral side such that the second ear stretch direction is oriented relative to the longitudinal direction at a second ear stretch angle when the absorbent article is oriented in the flat, laid out position and in the uncontracted state. In some embodiments, the first ear stretch angle and the second ear stretch angle are between 40° and 80°, inclusive.

According to certain embodiments, a method for manufacturing an absorbent article includes providing a chassis that extends in a longitudinal direction and includes a lateral side and providing an car formed of a fabric adapted to stretch in at least one of a machine direction or a cross-machine direction of the fabric such that the ear has a stretch direction that is parallel to one of the machine direction or the cross-machine direction of the fabric. The method further comprises attaching the car to the lateral side of the chassis such that the stretch direction of the ear is oriented relative to the longitudinal direction at an oblique angle when the absorbent article is oriented in a flat, laid-out position and in an uncontracted state.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 is a top plan view of an inner side of an absorbent article oriented in a flat, laid-out position and in an uncontracted state, according to one embodiment.

FIG. 1A is an enlarged section view taken at inset circle 1A in FIG. 1.

FIG. 2 is a top plan view of the opposing outer side of the absorbent article of FIG. 1.

FIG. 3 is a top plan view of the absorbent article of FIG. 1 with elastic members provided along some of the edges.

FIG. 4 is a front perspective view of the absorbent article of FIG. 3 positioned on a wearer.

FIG. 5 is a side perspective view of the absorbent article of FIG. 3 positioned on a wearer.

FIG. 6 is a rear perspective view of the absorbent article of FIG. 3 positioned on a wearer.

### DETAILED DESCRIPTION

The various aspects and embodiments of the invention will be described in the context of a reusable absorbent article, such as a reusable diaper or a semi-reusable diaper formed of a semi-durable chassis intended for disposal after multiple uses and cleaning (as opposed to disposable diapers intended for single use and reusable diapers intended for indefinite use). It is, however, readily apparent that the present invention could also be employed with other articles, such as disposable diapers, children's training pants, incontinence garments, and the like, which may be configured to be reusable or disposable. Typically, reusable articles, such as reusable garments, have a reusable component and an absorbent component (which can be reusable or disposable). In contrast, disposable articles, such as disposable garments, are intended for limited use and are not intended to be laundered or otherwise cleaned for reuse. A disposable diaper, for example, is discarded after it has become soiled by the wearer.

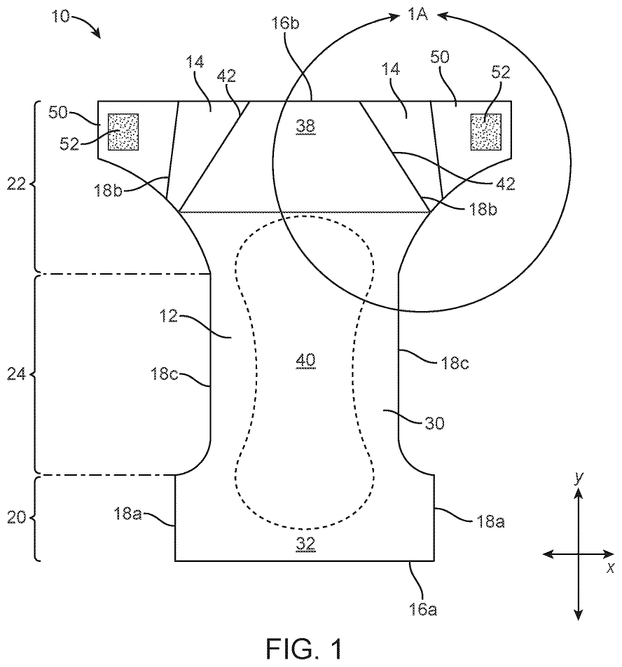

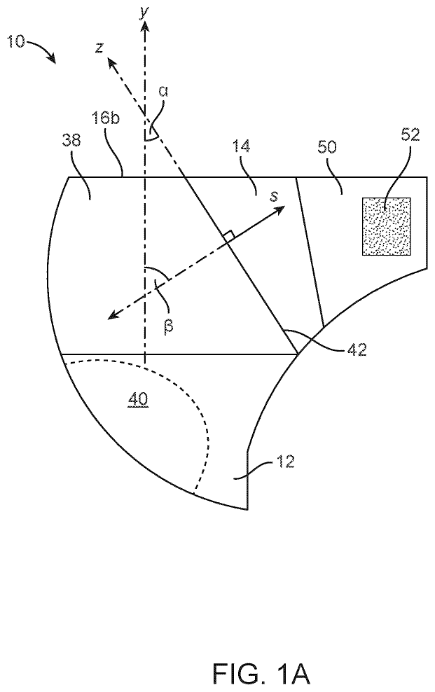

FIG. 1 is a plan view of an embodiment of an absorbent article (specifically a diaper 10) of the present invention in a flat, laid-out position and in an uncontracted state (i.e., with substantially all elastic induced gathering and contraction removed). Diaper 10 generally extends in a lateral direction x and a longitudinal direction y. The diaper 10 may have any desired shape or contour, such as rectangular, I-shaped, hourglass shape, or a T-shape. With the T-shape, the crossbar of the “T” may comprise the front waist region of the diaper, or may alternatively comprise the rear waist region of the diaper. The size or scale of the diaper 10 may be customized to the size of the wearer for which protection is desired or, alternatively, the diaper may be adjustable so as universally to fit wearers of different sizes.

In the illustrated embodiment, the diaper 10 includes a chassis 12 and ears 14. Generally, the outer edges of the chassis 12 define a periphery with generally laterally extending end edges connected by generally longitudinally extending side edges. The side edges define leg openings for the diaper 10, and optionally, are curvilinear and contoured.

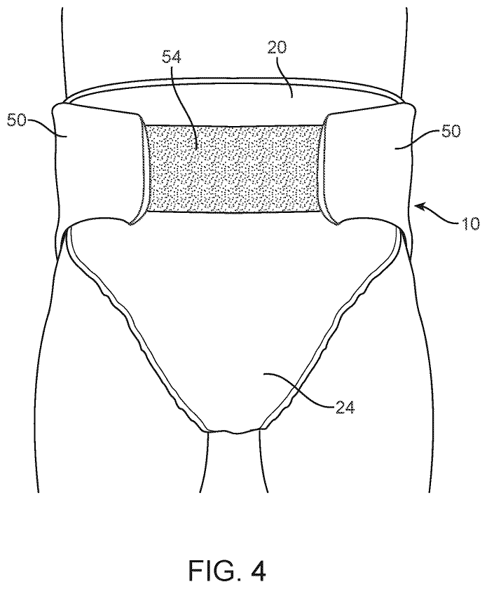

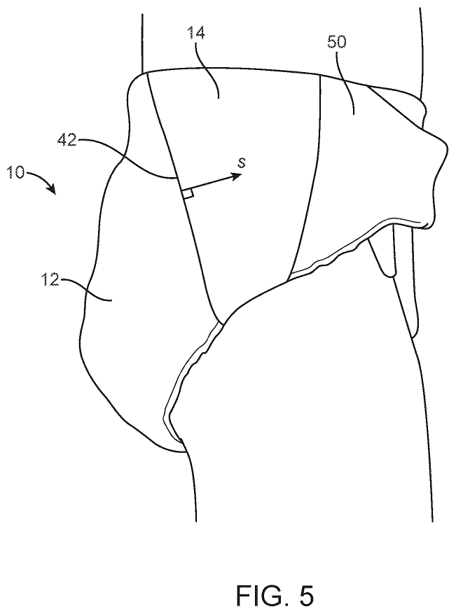

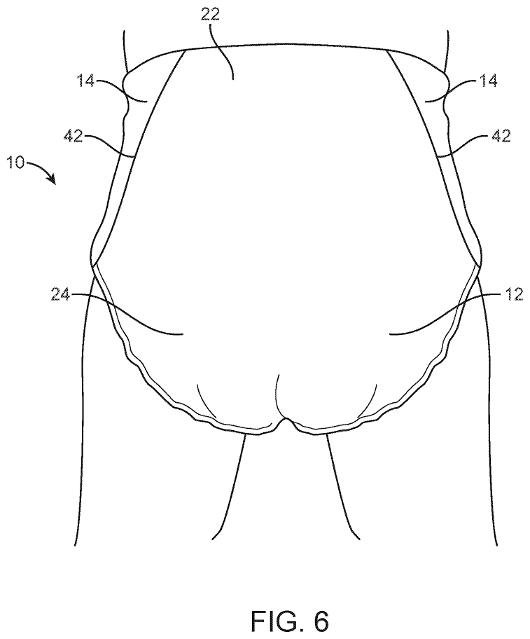

In the longitudinal direction y, the chassis 12 has a front waist region 20, a rear waist region 22, and an intermediate region 24 that connects the front waist region 20 and the rear waist region 22 and provides a crotch for placement between a wearer's legs. As shown in FIGS. 4-6 and in use, the intermediate region 24 is configured to be positioned to cover the crotch of the wearer, and the front waist region 20 and the rear waist region 22 are configured to extend at least partially around the waist of the wearer.

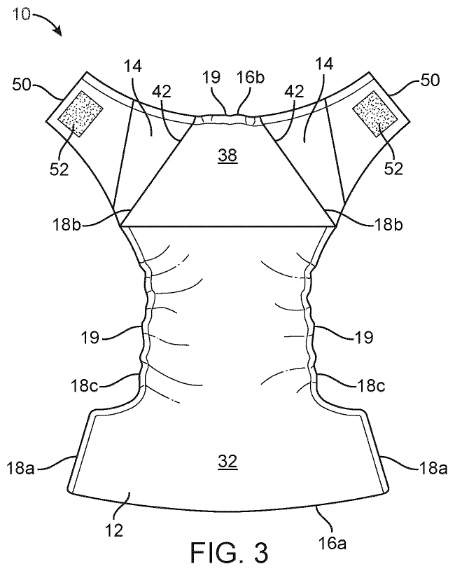

In the illustrated embodiment, the end edges include a front end edge 16a and a rear end edge 16b. The end edges 16a, 16b are shown as straight, but optionally, may be curvilinear. The side edges include laterally opposed front side edges 18a, laterally opposed rear side edges 18b, and laterally opposed intermediate side edges 18c that connect the front side edges 18a and the rear side edges 18b. In some embodiments, the front side edges 18a extend between the intermediate side edges 18c and the front end edge 16a, and the rear side edges 18b extend between the intermediate side edges 18c and the rear end edge 16b. In some embodiments, the rear side edges 18b taper inwardly from the intermediate side edges 18c towards the rear end edge 16b. Note, however, that FIG. 1 is merely representative and that the number and orientation of the end edges 16a, 16b and the side edges 18a-18c may vary depending on the particular article and its size and shape. Elastomeric members 19 (see FIG. 3) may be positioned to extend proximate or along any of the end edges 16a, 16b and/or side edges 18a-18c. The elastomeric members 19 draw and hold the diaper 10 against the wearer to better conform the diaper to the wearer and prevent leakage from the diaper.

While the chassis 12 may be formed as a single layer, in some embodiments the chassis is formed of at least two layers. Regardless and in some embodiments, the chassis 12 is formed of fabrics (e.g., woven, nonwoven, knitted, etc.) or other materials that are durable such that the diaper 10 may be cleaned (e.g., washed) and reused without damage.

By way only of example, in some embodiments the chassis 12 is formed of an outer shell fabric 26 having an exposed outer surface 28 and an inner liner fabric 30 having an exposed inner surface 32. The outer shell fabric 26 and inner liner fabric 30 may be attached to each other in different ways. In one non-limiting example, the outer shell fabric 26 and inner liner fabric 30 are joined via sewing or adhesive along at least a portion of the end edges 16a, 16b and/or the side edges 18a-18c.

In some embodiments, the outer shell fabric 26 is substantially liquid-impervious to thereby prevent moisture from penetrating through the chassis 12. Suitable materials include, but are not limited to, waterproof materials, water resistant materials, coated materials, laminated materials, etc. and, in some embodiments, may be woven, knitted, or nonwoven materials. Polymeric materials such as polyester, polyurethane, and/or elastomeric film laminates may be particularly suitable. The inner liner fabric 30 is located more proximate to, or directly in contact with, the wearer and thus may be formed of materials or in a way that imparts comfort but yet is still very durable. Such materials may be woven, knitted, or nonwoven and may include, but are not limited to, polymeric materials (e.g., polyester, polyurethane, etc.) and natural and synthetic cellulosic materials (e.g., cotton, rayon, acetate, triacetate, and lyocell fibers). In some embodiments, the inner liner fabric 30 is a fleece or microfleece material. In some embodiments, the inner liner fabric 30 may be a fabric capable of stretching.

One or more portions of the chassis 12 may include a reinforcing fabric element 38. By way only of example and with reference to FIG. 1, a reinforcing fabric element 38 has been provided in the rear waist region 22 but could be provided in alternative and/or additional regions of the chassis 12. While the reinforcing fabric element 38 may be a non-stretch material, in some embodiments the reinforcing fabric element 38 is formed of a stretch fabric that enhances the conformity and fit of the diaper around the wearer. For example, the reinforcing fabric element 38 may be a woven or knit fabric that is able to stretch and recover in at least one (or both) of the machine direction or cross-machine direction of the fabric. In some embodiments, the reinforcing fabric element 38 is formed from natural and synthetic cellulosic material (e.g., cotton, rayon, acetate, triacetate, and lyocell fibers) to impart comfort, nylon material to impart abrasion resistance and durability, and/or an elastomeric material (e.g., as spandex (elastane), ethylene-olefin copolymer, rubber, etc.) to impart stretch properties. In one specific, non-limiting embodiment, the reinforcing fabric element 38 is a fabric formed of 60% rayon, 33% nylon, and 7% spandex (e.g., Lycra®).

An absorbent component 40 (shown schematically in FIG. 1) is provided on and/or within at least a portion of the chassis 12 to be available to absorb and wick exudates and moisture away from the wearer. The absorbent component 40 can be formed of any number of shapes and sizes and can include superabsorbent material, staple fibers, binders, and the like, and combinations thereof as is known in the art. For example, the absorbent component 40 may be formed of any suitable material or assembly of materials, including, but not limited to, those disclosed in U.S. Pat. No. 9,011,403, issued Apr. 21, 2015 and entitled “Absorbent Insert”; U.S. Pat. No. 6,710,225, issued Mar. 23, 2004 and entitled “Layered Absorbent Structure with a Zoned Basis Weight”; U.S. application Ser. No. 16/086,033, published as US 2020/0289342 on Sep. 17, 2020 and entitled “Absorbent Cores and Methods for Forming Absorbent Cores”; and PCT/US2020/047736, published as WO 2022/046030 on Mar. 3, 2022 and entitled “Absorbent Structures and Methods for Manufacturing Absorbent Structures,” the entirety of each of which is herein incorporated by reference. In the context of reusable diapers, the absorbent component 40 may be a disposable insert that is removed after use and replaced within the chassis 12 by a new disposable insert, a reusable insert that is cleaned after use and then replaced within the chassis 12, and/or permanently incorporated on or into the chassis 12 such that both the chassis 12 with associated absorbent component 40 are cleaned and reused after use. The absorbent component 40 may be retained within the chassis 12 in myriad ways, such as via insertion into a pocket within the chassis 12 or via mechanical or chemical retention features (e.g., hook and loop fasteners, snaps, adhesives, etc.).

Opposing ears 14 are attached to the rear waist region 22 of the chassis 12 at one or more attachment points to extend outwardly from the chassis 12. The ears 14 may be attached to the chassis 12 using any suitable mechanical or chemical attachment method, including, but not limited to, sewing/stitching, adhering, melting, etc.

As best seen in FIG. 1A, in some embodiments the ears 14 are attached to the rear side edges 18b of the chassis 12 along bond lines 42 that extend along a bond line axis z oriented at a bond angle α relative to the longitudinal direction y of the diaper 10. In some embodiments, the bond angle α extends at an oblique angle relative to the longitudinal direction y (i.e., at an angle that is greater than 0° and less than 90°). In some embodiments, the bond angle α is between 5° to 85°, inclusive; between 10° and 80°, inclusive; between 15° and 75°, inclusive; between 15° and 70°, inclusive; between 20° and 65°, inclusive; between 20° and 60°, inclusive; between 20° and 55°, inclusive; between 20° and 50°, inclusive; between 20° and 45°, inclusive; between 20° and 40°, inclusive; between 20° and 35°, inclusive; between 20° and 30°, inclusive; between 15° and 40°, inclusive; between 15° and 35°, inclusive; and between 15° and 30°, inclusive.

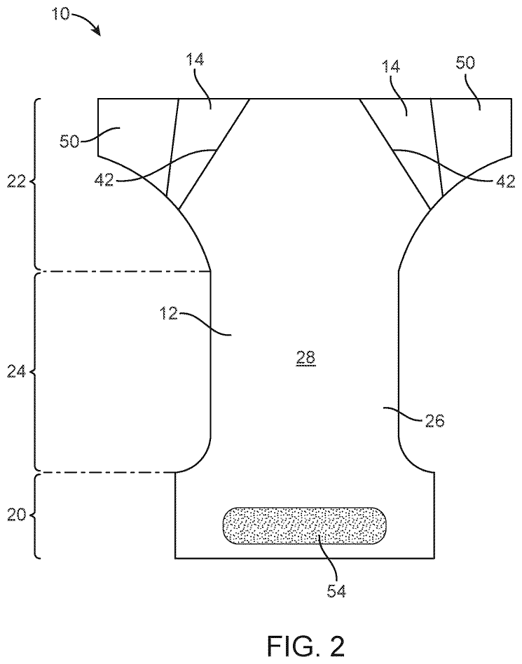

For purposes of the present disclosure, the bond angle α (as well as the stretch angle β introduced below) is measured when the diaper 10 is in a flat, laid-out position and in an uncontracted state (i.e., with substantially all elastic induced gathering and contraction removed either physically or via stretching), such as shown in FIGS. 1 and 2. Traditionally, ears have been attached to a diaper chassis along bond lines that extend parallel to the longitudinal direction of the chassis. Subsequent contraction of elastic members in the diaper (such as along the side edges and waistband) draws the chassis inwardly, creating the appearance that the bond lines extend at an oblique angle relative to the longitudinal direction when really they do not.

Ears 14 may be made of any suitable material but, in some embodiments, are made from a woven or knit fabric that is able to stretch and recover in at least one (or both) of the machine direction or cross-machine direction of the fabric. For example, in some embodiments, the ear fabric is formed from one or more of natural and synthetic cellulosic material (e.g., cotton, rayon, acetate, triacetate, and lyocell fibers) to impart comfort, nylon material to impart abrasion resistance and durability, and/or an elastomeric material (e.g., as spandex (elastane), ethylene-olefin copolymer, rubber, etc.) to impart stretch properties. In one specific, non-limiting embodiment, the ear fabric is formed of 60% rayon, 33% nylon, and 7% spandex (e.g., Lycra®). In some embodiments, the stretch fabric used to form ears 14 is the same as that which can form the reinforcing fabric element 38.

In some embodiments, the ears 14 are attached such that the stretch direction s (i.e., the orientation of stretch of the ear 14, which will typically be the machine and/or cross-machine direction(s) of the ear fabric) extends at a stretch angle β relative to the longitudinal direction y. In some embodiments, the stretch angle β extends at an oblique angle relative to the longitudinal direction y (i.e., greater than 0° and less than 90°). In some embodiments, the stretch angle β is between 5° to 85°, inclusive; between 10° and 80°, inclusive; between 15° and 80°, inclusive; between 20° and 80°, inclusive; between 25° and 80°, inclusive; between 30° and 80°, inclusive; between 40° and 80°, inclusive; between 40° and 75°, inclusive; between 40° and 70°, inclusive; between 45° and 75°, inclusive; between 45° and 70°, inclusive; between 50° and 75°, inclusive; and between 50° and 70°, inclusive.

In some embodiments, the stretch angle β is substantially perpendicular to the bond line axis z, but such is not a requirement. In some embodiments, the ear 14 is oriented such that the direction of maximum or greatest stretch of the fabric (e.g., the cross-machine direction) extends at the stretch angle β relative to the longitudinal direction y.

Securing tabs 50 provided with securing features may be attached to the ears 14 distal the chassis 12. The securing tabs 50 may be formed of any material and in some embodiment may be formed of the same material as the chassis 12 or of a stretch material such as that used to form cars 14. Any type of securing features may be provided on the securing tabs 50 to cooperate with complimentary securing features on the chassis 12 to secure the diaper 10 around the waist of the wearer. Examples include, but are not limited to, book-and-loop fasteners, adhesives, snaps, clips, buttons, clasps, magnets, adhesive fasteners, cohesive fasteners, mechanical fasteners, male/female mating components, combinations thereof, and/or other suitable features as desired. It may be preferable that the securing features do not create a permanent bond but rather permit repeatable fastening of the diaper 10 on the wearer. In one, non-limiting embodiment, the securing features are formed of securing patches 52 of hooks (or loops) that engage a securing band 54 formed of loops (or hooks) provided on the exposed outer surface 28 of the front waist region 20 of the chassis 12 to permit adjustable positioning of the securing patches 52 on the securing band 54 to accommodate wearers of different sizes. In some embodiments, the diaper 10 does not include separate securing tabs 50. Rather, the securing features (e.g., securing patches 52) are provided directly on the ears 14.

In use, the diaper 10 is positioned between the legs of the wearer with the front waist region 20 proximate the anterior of the wearer and the rear waist region 22 proximate the posterior of the wearer. The ears 14 may be stretched to wrap around a portion of the wearer (e.g., laterally around the hips), and the diaper 10 is secured via the securing features (e.g., securing patches 52 and securing band 54) such that the rear waist region 22 is secured to the front waist region 20 of the chassis 12 to retain the diaper 10 on the wearer. The ability of the cars 14 to stretch permits adjustability of the diaper's waist size such that the diaper's waist size can be tailored to the wearer's waist size to provide a snug, contoured fit around the wearer. The ability of the diaper 10 to contour to the wearer is further enhanced by the angled relationship between the stretch direction s of the ears 14 and the longitudinal direction y of the diaper 10. As explained above, traditionally ears have been attached to a diaper chassis along bond lines that extend parallel to the longitudinal direction of the diaper and such that the ears stretch (to the extent they do) directly outwardly (i.e., at a 90° angle) relative to the longitudinal direction of the diaper. While this design permits the ears to extend laterally around the hips, it fails to account for the upwardly curving nature of the hips. By attaching stretchable ears 14 to the chassis 12 such that the stretch direction s is oblique to the longitudinal direction y, the ears 14 are permitted to stretch upwardly as well as outwardly from the chassis 12. In this way, the ears 14 can extend up, over, and around the hips (as opposed to just laterally around the hips) to better contour the diaper 10 to the wearer's bottom and to more effectively stretch the elastic around the hips, legs and waist of the wearer to provide a tighter seal between the diaper 10 and the wearer. This results is a better, more comfortable fit that results in less droop (downward movement) and consequent leakage from the diaper. As best seen in FIGS. 4-6, the ears extend up and over the hips of the wearer so as to at least partially cover the lateral sides of the wearer's hips.

Embodiments of the invention also relate to a method for manufacturing an absorbent article (such as, but not limited to, embodiments of the diaper 10 described above) having a chassis and at least one ear formed of a fabric that stretches in at least one stretch direction. In accordance with the method, the ear is attached to the chassis such that the stretch direction of the car (and in some embodiments, the direction of maximum stretch) extends at a stretch angle that is oblique relative to a longitudinal direction of the chassis and, in some embodiments, that is at the stretch angles β disclosed above. In some embodiments of the method, the ear is attached to the chassis along a bond line that extends along a bond line axis that is oriented at a bond angle that is oblique relative to the longitudinal direction and, in some embodiments, at the bond angles α disclosed above. In some embodiments, the ear is attached such that the stretch direction is substantially perpendicular to the bond line axis. The method may further comprise incorporating securing features into the diaper. In some embodiments, the securing features are provided on securing tabs that are attached to the ears opposite the chassis or directly on the ears themselves.

### EXAMPLES

A collection of exemplary embodiments, including at least some explicitly enumerated as “Examples” providing additional description of a variety of example types in accordance with the concepts described herein are provided below. These examples are not meant to be mutually exclusive, exhaustive, or restrictive; and the invention is not limited to these examples but rather encompasses all possible modifications and variations within the scope of the issued claims and their equivalents.
Example 1. An absorbent article comprising a chassis extending in a longitudinal direction and comprising a front portion, a rear portion, an intermediate portion connecting the front portion and the rear portion, a first lateral side, an opposing second lateral side, an outer surface and an opposing inner surface; and at least one ear attached to, and extending outwardly from, the first lateral side of the chassis, wherein the at least one ear is formed of a fabric adapted to stretch such that the at least one ear has a stretch direction and wherein the at least one ear is attached to the first lateral side such that the stretch direction of the at least one ear is oriented relative to the longitudinal direction at an oblique stretch angle when the absorbent article is oriented in a flat, laid-out position and in an uncontracted state.
Example 2. The absorbent article of any of the preceding or subsequent examples or combination of examples, wherein the at least one ear comprises a first ear and a second ear, each of the first ear and the second ear formed of the fabric and having the stretch direction, wherein the first ear and the second ear are respectively attached to the first lateral side and the second lateral side such that the stretch direction of each of the first ear and the second ear is oriented relative to the longitudinal direction at an oblique angle when the absorbent article is oriented in the flat, laid-out position and in the uncontracted state.
Example 3. The absorbent article of any of the preceding or subsequent examples or combination of examples, wherein the fabric has a machine direction and a cross-machine direction and wherein the stretch direction is parallel to one of the machine direction or the cross-machine direction.
Example 4. The absorbent article of any of the preceding or subsequent examples or combination of examples, wherein the oblique stretch angle is between 40° and 80°, inclusive.
Example 5. The absorbent article of any of the preceding or subsequent examples or combination of examples, wherein the at least one ear is attached to the chassis along a bond line having a bond line axis, wherein the bond line axis extends relative to the longitudinal direction at an oblique bond angle when the absorbent article is oriented in the flat, laid-out position and in the uncontracted state.
Example 6. The absorbent article of any of the preceding or subsequent examples or combination of examples, wherein the oblique bond angle is between 20° and 50°, inclusive.
Example 7. The absorbent article of any of the preceding or subsequent examples or combination of examples, wherein the stretch direction is substantially perpendicular to the bond line axis.
Example 8. The absorbent article of any of the preceding or subsequent examples or combination of examples, wherein the at least one ear is attached to the rear portion of the chassis.
Example 9. The absorbent article of any of the preceding or subsequent examples or combination of examples, wherein the fabric comprises a woven or knitted fabric comprising elastane.
Example 10. The absorbent article of any of the preceding or subsequent examples or combination of examples, further comprising an absorbent component positioned on or within the chassis.
Example 11. The absorbent article of any of the preceding or subsequent examples or combination of examples, wherein the absorbent article comprises a diaper.
Example 12. The absorbent article of any of the preceding or subsequent examples or combination of examples, wherein the diaper is a reusable diaper and wherein the chassis is adapted to be washed and reused.
Example 13. The absorbent article of any of the preceding or subsequent examples or combination of examples, further comprising a first securing feature and a second securing feature, wherein the first securing feature is associated with the at least one ear and the second securing feature is provided on the outer surface of the front portion of the chassis, wherein the first securing feature and the second securing feature are configured to cooperate with each other to secure the absorbent article on a wearer.
Example 14. The absorbent article of any of the preceding or subsequent examples or combination of examples, further comprising a securing tab attached to the at least one ear, wherein the first securing feature is provided on the securing tab.
Example 15. An absorbent article comprising a chassis extending in a longitudinal direction and comprising a front portion, a rear portion, an intermediate portion connecting the front portion and the rear portion, a first lateral side, an opposing second lateral side, an outer surface and an opposing inner surface; a first ear attached to, and extending outwardly from, the first lateral side; and a second ear attached to, and extending outwardly from, the second lateral side, wherein the first ear and the second ear are each formed of a fabric adapted to stretch in at least one of a machine direction or a cross-machine direction such that the first ear has a first ear stretch direction and the second ear has a second ear stretch direction, wherein each of the first ear stretch direction and the second ear stretch direction is parallel to one of the machine direction or the cross-machine direction of the fabric; wherein the first ear is attached to the first lateral side such that the first ear stretch direction is oriented relative to the longitudinal direction at a first ear stretch angle when the absorbent article is oriented in a flat, laid-out position and in an uncontracted state; wherein the second ear is attached to the second lateral side such that the second ear stretch direction is oriented relative to the longitudinal direction at a second ear stretch angle when the absorbent article is oriented in the flat, laid-out position and in the uncontracted state; and wherein the first ear stretch angle and the second ear stretch angle are between 40° and 80°, inclusive.
Example 16. The absorbent article of any of the preceding or subsequent examples or combination of examples, wherein the first ear is attached to the chassis along a first bond line and the second ear is attached to the chassis along a second bond line, wherein each of the first bond line and the second bond line extends at an oblique bond line angle relative to the longitudinal direction when the absorbent article is oriented in the flat, laid-out position and in the uncontracted state.
Example 17. The absorbent article of any of the preceding or subsequent examples or combination of examples, wherein the first ear stretch direction is substantially perpendicular to the first bond line and the second ear stretch direction is substantially perpendicular to the second bond line.
Example 18. The absorbent article of any of the preceding or subsequent examples or combination of examples, wherein the first ear and the second ear are attached to the rear portion of the chassis.
Example 19. The absorbent article of any of the preceding or subsequent examples or combination of examples, wherein the absorbent article comprises a diaper and the diaper further comprises an absorbent component positioned on or within the chassis.
Example 20. A method for manufacturing an absorbent article comprising providing a chassis that extends in a longitudinal direction and comprises a lateral side; providing an ear formed of a fabric adapted to stretch in at least one of a machine direction or a cross-machine direction of the fabric such that the ear has a stretch direction that is parallel to the one of the machine direction or the cross-machine direction of the fabric; and attaching the ear to the lateral side of the chassis such that the stretch direction of the ear is oriented relative to the longitudinal direction at an oblique angle when the absorbent article is oriented in a flat, laid-out position and in an uncontracted state.

The various aspects, embodiments, implementations or features of the described embodiments can be used separately or in any combination. It should be appreciated that the various elements of concepts from the figures may be combined without departing from the spirit or scope of the invention.

The use of the terms “a” and “an” and “the” and similar referents in the context of describing the invention (especially in the context of the following claims) are to be construed to cover both the singular and the plural, unless otherwise indicated herein or clearly contradicted by context. Directional references such as “up,” “down,” “top,” “bottom,” “left,” “right,” “front,” and “back,” among others, are intended to refer to the orientation as illustrated and described in the figure (or figures) to which the components and directions are referencing. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, or gradients thereof, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, the nearness of completion will be so as to have the same overall result as if absolute and total completion were obtained.

Preferred embodiments of this invention are described herein, including the best mode known to the inventors for carrying out the invention. The invention is susceptible to various modifications and alternative constructions, and certain shown exemplary embodiments thereof are shown in the drawings and have been described above in detail. Variations of those preferred embodiments, within the spirit of the present invention, may become apparent to those of ordinary skill in the art upon reading the foregoing description. The inventors expect skilled artisans to employ such variations as appropriate, and the inventors intend for the invention to be practiced otherwise than as specifically described herein. Accordingly, it should be understood that there is no intention to limit the invention to the specific form or forms disclosed, but on the contrary, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It will be apparent to one of ordinary skill in the art that many modifications and variations are possible in view of the above teachings.

We claim:

1. An absorbent article comprising:
a chassis extending in a longitudinal direction and comprising a front portion, a rear portion, an intermediate portion connecting the front portion and the rear portion, a first lateral side, an opposing second lateral side, an outer surface and an opposing inner surface; and
at least one ear attached to, and extending outwardly from, the first lateral side of the chassis,
wherein:
the at least one ear is formed of a fabric adapted to stretch such that the at least one ear has a stretch direction; and
the at least one ear is attached to the first lateral side such that the stretch direction of the at least one ear is oriented relative to the longitudinal direction at an oblique stretch angle when the absorbent article is oriented in a flat, laid-out position and in an uncontracted state.

2. The absorbent article of claim 1, wherein the at least one ear comprises a first ear and a second ear, each of the first ear and the second ear formed of the fabric and having the stretch direction, wherein the first ear and the second ear are respectively attached to the first lateral side and the second lateral side such that the stretch direction of each of the first ear and the second ear is oriented relative to the longitudinal direction at an oblique angle when the absorbent article is oriented in the flat, laid-out position and in the uncontracted state.

3. The absorbent article of claim 1, wherein the fabric has a machine direction and a cross-machine direction and wherein the stretch direction is parallel to one of the machine direction or the cross-machine direction.

4. The absorbent article of claim 1, wherein the oblique stretch angle is between 40° and 80°, inclusive.

5. The absorbent article of claim 1, wherein the at least one ear is attached to the chassis along a bond line having a bond line axis, wherein the bond line axis extends relative to the longitudinal direction at an oblique bond angle when the absorbent article is oriented in the flat, laid-out position and in the uncontracted state.

6. The absorbent article of claim 5, wherein the oblique bond angle is between 20° and 50°, inclusive.

7. The absorbent article of claim 5, wherein the stretch direction is substantially perpendicular to the bond line axis.

8. The absorbent article of claim 1, wherein the at least one ear is attached to the rear portion of the chassis.

9. The absorbent article of claim 1, wherein the fabric comprises a woven or knitted fabric comprising elastane.

10. The absorbent article of claim 1, further comprising an absorbent component positioned on or within the chassis.

11. The absorbent article of claim 10, wherein the absorbent article comprises a diaper.

12. The absorbent article of claim 11, wherein the diaper is a reusable diaper and wherein the chassis is adapted to be washed and reused.

13. The absorbent article of claim 1, further comprising a first securing feature and a second securing feature, wherein the first securing feature is associated with the at least one ear and the second securing feature is provided on the outer surface of the front portion of the chassis, wherein the first securing feature and the second securing feature are configured to cooperate with each other to secure the absorbent article on a wearer.

14. The absorbent article of claim 13, further comprising a securing tab attached to the at least one ear, wherein the first securing feature is provided on the securing tab.

15. An absorbent article comprising:
a chassis extending in a longitudinal direction and comprising a front portion, a rear portion, an intermediate portion connecting the front portion and the rear portion, a first lateral side, an opposing second lateral side, an outer surface and an opposing inner surface;
a first ear attached to, and extending outwardly from, the first lateral side; and
a second ear attached to, and extending outwardly from, the second lateral side,
wherein:
the first ear and the second ear are each formed of a fabric adapted to stretch in at least one of a machine direction or a cross-machine direction such that the first ear has a first ear stretch direction and the second ear has a second ear stretch direction, wherein each of the first ear stretch direction and the second ear stretch direction is parallel to one of the machine direction or the cross-machine direction of the fabric;
the first ear is attached to the first lateral side such that the first ear stretch direction is oriented relative to the longitudinal direction at a first ear stretch angle when the absorbent article is oriented in a flat, laid-out position and in an uncontracted state;
the second ear is attached to the second lateral side such that the second ear stretch direction is oriented relative to the longitudinal direction at a second ear stretch angle when the absorbent article is oriented in the flat, laid-out position and in the uncontracted state; and
the first ear stretch angle and the second ear stretch angle are between 40° and 80°, inclusive.

16. The absorbent article of claim 15, wherein the first ear is attached to the chassis along a first bond line and the second ear is attached to the chassis along a second bond line, wherein each of the first bond line and the second bond line extends at an oblique bond line angle relative to the longitudinal direction when the absorbent article is oriented in the flat, laid-out position and in the uncontracted state.

17. The absorbent article of claim 16, wherein the first ear stretch direction is substantially perpendicular to the first bond line and the second ear stretch direction is substantially perpendicular to the second bond line.

18. The absorbent article of claim 15, wherein the first ear and the second ear are attached to the rear portion of the chassis.

19. The absorbent article of claim 15, wherein the absorbent article comprises a diaper and the diaper further comprises an absorbent component positioned on or within the chassis.

20. A method for manufacturing an absorbent article comprising:
providing a chassis that extends in a longitudinal direction and comprises a lateral side;
providing an ear formed of a fabric adapted to stretch in at least one of a machine direction or a cross-machine direction of the fabric such that the ear has a stretch direction that is parallel to the one of the machine direction or the cross-machine direction of the fabric; and
attaching the ear to the lateral side of the chassis such that the stretch direction of the ear is oriented relative to the longitudinal direction at an oblique angle when the absorbent article is oriented in a flat, laid-out position and in an uncontracted state.
